(19) United States
(12) Patent Application Publication
(10) Pub. No.: US 2012/0237932 A1
(43) Pub. Date: Sep. 20, 2012

(54) METHODS AND MATERIALS FOR ASSESSING THE CIS/TRANS NATURE OF HUMANS HAVING CYP2C19*2 AND CYP2C19*17 ALLELES

(76) Inventors: Jennifer Skierka, Oronoco, MN (US); Dennis J. O'Kane, Rochester, MN (US); John L. Black, Rochester, MN (US)

(21) Appl. No.: 13/420,252

(22) Filed: Mar. 14, 2012

Related U.S. Application Data
(60) Provisional application No. 61/452,886, filed on Mar. 15, 2011.

(51) Int. Cl. C12Q 1/68

(52) U.S. Cl. 435/6.11

(57) ABSTRACT

This document provides methods and materials involved in determining if a human heterozygous for CYP2C19*2 and heterozygous for CYP2C19*17 contains one CYP2C19 allele with both CYP2C19*2 and CYP2C19*17 (e.g., a cis relationship) or contains one CYP2C19 allele with CYP2C19*2 and one CYP2C19 allele with CYP2C19*17 (e.g., a trans relationship).

### CROSS-REFERENCE TO RELATED APPLICATIONS

This application claims the benefit of U.S. Provisional Application Ser. No. 61/452,886, filed Mar. 15, 2011. The disclosure of the prior application is considered part of (and is incorporated by reference in) the disclosure of this application.

### BACKGROUND

1. Technical Field

This document relates to methods and materials involved in determining if a human heterozygous for CYP2C19*2 and heterozygous for CYP2C19*17 contains one allele with both CYP2C19*2 and CYP2C19*17 (e.g., a cis relationship) or contains one allele with CYP2C19*2 and the other allele with CYP2C19*17 (e.g., a trans relationship).

2. Background Information

Cytochrome P450, family 2, subfamily C, polypeptide 19 (CYP2C19) is a member of the cytochrome P450 mixed-function oxidase system. CYP2C19 can metabolize several important groups of drugs including proton pump inhibitors and anti-epileptics. CYP2C19 has been annotated as (R)-limonene 6-monooxygenase and (S)-limonene 6-monooxygenase. The gene encoding CYP2C19 is highly variable, and may be duplicated, deleted, partially deleted, or have a host of polymorphisms. The genotype or allele type can account for CYP2C19 function, which can be normal, enhanced, reduced, or non-existent.

### SUMMARY

This document provides methods and materials involved in determining if a human heterozygous for CYP2C19*2 and heterozygous for CYP2C19*17 contains one CYP2C19 allele with both CYP2C19*2 and CYP2C19*17 (e.g., a cis relationship) or contains one CYP2C19 allele with CYP2C19*2 and one CYP2C19 allele with CYP2C19*17 (e.g., a trans relationship). For example, sequencing techniques can be used to assess a strand of nucleic acid for the presence of a mutant nucleotide (i.e., T, A, or G) or a wild-type nucleotide (i.e., C) at the CYP2C19*17 position (i.e., position c.-806) together with the presence of a mutant nucleotide (i.e., C, A, or G) or a wild-type nucleotide (i.e., T) at the c.-98 position. The presence of both a mutant nucleotide at the CYP2C19*17 position and a mutant nucleotide at the c.-98 position on a single nucleic acid strand can indicate that the human contains CYP2C19*2 and CYP2C19*17 in a cis relationship. In other words, the human contains a CYP2C19 allele with both CYP2C19*2 and CYP2C19*17. Likewise, given the fact that the human is heterozygous for CYP2C19*2 and heterozygous for CYP2C19*17, the presence of both a wild-type nucleotide at the CYP2C19*17 position and a wild-type nucleotide at the c.-98 position can indicate that the human contains CYP2C19*2 and CYP2C19*17 in a cis relationship. In other words, the human contains a CYP2C19 allele with both CYP2C19*2 and CYP2C19*17. The presence of a wild-type nucleotide at the CYP2C19*17 position and a mutant nucleotide at the c.-98 position or the presence of a mutant nucleotide at the CYP2C19*17 position and a wild-type nucleotide at the c.-98 position can indicate that the human contains CYP2C19*2 and CYP2C19*17 in a trans relationship. In other words, the human contains one CYP2C19 allele with CYP2C19*2 and one CYP2C19 allele with CYP2C19*17.

Having the ability to determine the cis or trans relationship of CYP2C19*2 and CYP2C19*17 within a human heterozygous for CYP2C19*2 and heterozygous for CYP2C19*17 can aid in patient care because CYP2C19 polypeptides can regulate the activation or inactivation of many drugs in common use across all medical specialties. For example, humans heterozygous for CYP2C19*2 and heterozygous for CYP2C19*17 in a cis relationship can experience less CYP2C19-mediated metabolism than humans heterozygous for CYP2C19*2 and heterozygous for CYP2C19*17 in a trans relationship. Knowledge of CYP2C19 genetic arrangements and methodologies for detecting such arrangements can allow genotypes to be interpreted correctly which, in turn, can have a significant impact on patient care.

In general, one aspect of this document features a method for assessing the cis or trans nature of a human heterozygous for CYP2C19*2 and heterozygous for CYP2C19*17. The method comprises, or consists essentially of, (a) determining if a CYP2C19 allele of the human comprises (i) a T, A, or G at the c. 806 position and a C, A, or G at the c.-98 position, (ii) a C at the c.-806 position and a T at the c.-98 position, (iii) a C at the c.-806 position and a C, A, or G at the c.-98 position, or (iv) a T, A, or G at the c. 806 position and a T at the c.-98 position, (b) classifying the human as having CYP2C19*2 and CYP2C19*17 in cis if the CYP2C19 allele comprises (i) or (ii), and (c) classifying the human as having CYP2C19*2 and CYP2C19*17 in trans if the CYP2C19 allele comprises (iii) or (iv). The human can be a cardiovascular disease patient. The CYP2C19 allele can comprise a T, A, or G at the c. 806 position and a C, A, or G at the c.-98 position, and the human can be classified as having CYP2C19*2 and CYP2C19*17 in cis. The CYP2C19 allele can comprise a T at the c.-806 position and a C at the c.-98 position, and the human can be classified as having CYP2C19*2 and CYP2C19*17 in cis. The CYP2C19 allele can comprise a C at the c.-806 position and a T at the c.-98 position, and the human can be classified as having CYP2C19*2 and CYP2C19*17 in cis. The CYP2C19 allele can comprise a C at the c.-806 position and a C, A, or G at the c.-98 position, and the human can be classified as having CYP2C19*2 and CYP2C19*17 in trans. The CYP2C19 allele can comprise a C at the c.-806 position and a C at the c.-98 position, and the human can be classified as having CYP2C19*2 and CYP2C19*17 in trans. The CYP2C19 allele can comprise a T, A, or G at the c.-806 position and a T at the c.-98 position, and the human can be classified as having CYP2C19*2 and CYP2C19*17 in trans. The CYP2C19 allele can comprise a T at the c.-806 position and a T at the c.-98 position, and the human can be classified as having CYP2C19*2 and CYP2C19*17 in trans.

In another aspect, this document features a method for identifying a human heterozygous for CYP2C19*2 and heterozygous for CYP2C19*17 having CYP2C19*2 and CYP2C19*17 in cis. The method comprises, or consists essentially of, (a) detecting the presence of an CYP2C19 allele of the human comprising (i) a T, A, or G at the c. 806 position and a C, A, or G at the c.-98 position or (ii) a C at the c.-806 position and a T at the c.-98 position, and (b) classifying the human as having CYP2C19*2 and CYP2C19*17 in cis based at least in part on the presence of the CYP2C19 allele. The human can be a cardiovascular disease patient. The CYP2C19 allele can comprise a T, A, or G at the c. 806 position and a C, A, or G at the c.-98 position. The CYP2C19 allele can comprise a T at the c.-806 position and a C at the c.-98 position. The CYP2C19 allele can comprise a C at the c.-806 position and a T at the c.-98 position.

In another aspect, this document features a method for identifying a human heterozygous for CYP2C19*2 and heterozygous for CYP2C19*17 having CYP2C19*2 and CYP2C19*17 in trans. The method comprises, or consisting essentially of, (a) detecting the presence of an CYP2C19 allele of the human comprising (i) a C at the c.-806 position and a C, A, or G at the c.-98 position or (ii) a T, A, or G at the c. 806 position and a T at the c.-98 position, and (b) classifying the human as having CYP2C19*2 and CYP2C19*17 in trans based at least in part on the presence of the CYP2C19 allele. The human can be a cardiovascular disease patient. The CYP2C19 allele can comprise a C at the c.-806 position and a C, A, or G at the c.-98 position. The CYP2C19 allele can comprise a C at the c.-806 position and a C at the c.-98 position. The CYP2C19 allele can comprise a T, A, or G at the c.-806 position and a T at the c.-98 position. The CYP2C19 allele can comprise a T at the c.-806 position and a T at the c.-98 position.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a diagram of a CYP2C19 allele showing the location of PCR primers, sequencing primers, *17 (c.-806), c.-98, and *4 (c.1).

FIG. 2 is a diagram of CYP2C19 alleles when CYP2C19*2 and CYP2C19*17 are in a cis relationship for a human heterozygous for CYP2C19*2 and heterozygous for CYP2C19*17.

FIG. 3 is a diagram of CYP2C19 alleles when CYP2C19*2 and CYP2C19*17 are in a trans relationship for a human heterozygous for CYP2C19*2 and heterozygous for CYP2C19*17.

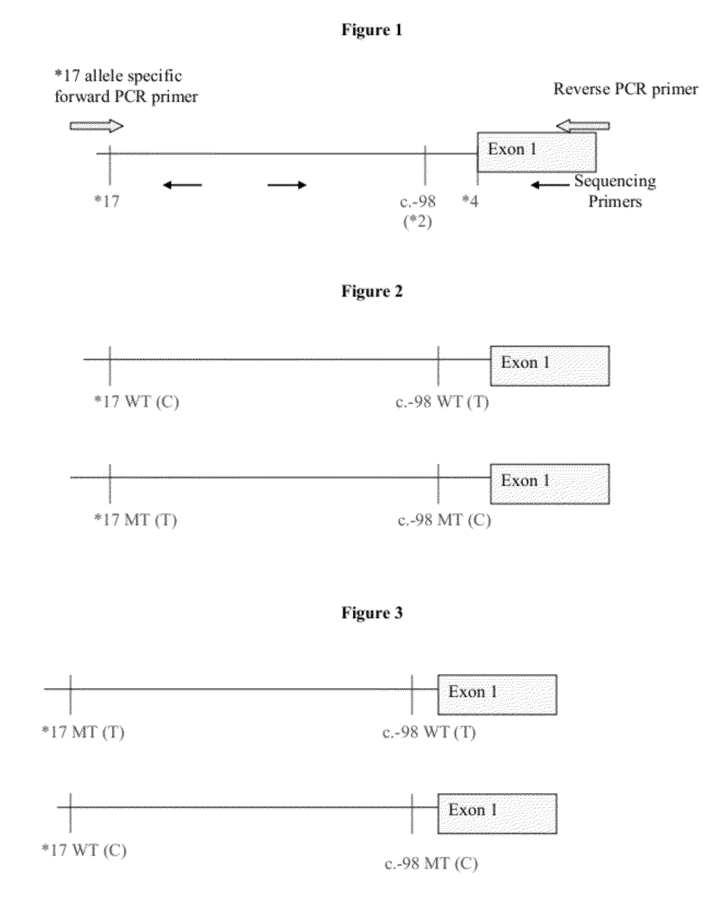

### DETAILED DESCRIPTION

This document provides methods and materials involved in determining if a human heterozygous for CYP2C19*2 and heterozygous for CYP2C19*17 contains one CYP2C19 allele with both CYP2C19*2 and CYP2C19*17 (e.g., a cis relationship) or contains one CYP2C19 allele with CYP2C19*2 and one CYP2C19 allele with CYP2C19*17 (e.g., a trans relationship). For example, sequencing techniques can be used to assess a strand of nucleic acid for the presence of a mutant nucleotide (i.e., T, A, or G) or a wild-type nucleotide (i.e., C) at the CYP2C19*17 position (i.e., position c.-806) together with the presence of a mutant nucleotide (i.e., C, A, or G) or wild-type nucleotide (i.e., T) at the c.-98 position. Since the presence of a wild-type nucleotide (i.e., T) at the c.-98 position indicates that that allele contains a wild-type nucleotide (i.e., G) at the CYP2C19*2 position (i.e., position c.681) and the presence of a mutant nucleotide (i.e., C, A, or G) at the c.-98 position indicates that that allele contains a mutant nucleotide (i.e., C, A, or T) at the CYP2C19*2 position (i.e., position c.681), an assessment of the c.-98 position can be performed with an assessment of the CYP2C19*17 position to determine if CYP2C19*2 and CYP2C19*17 are in a cis or trans relationship. A CYP2C19 nucleic acid sequence can be found in an NCBI database (e.g., on the world wide web at “ncbi.nlm.nih.gov”). For example, a human CYP2C19 nucleic acid can be as set forth in GenBank® GI No. 224589801 (Accession No. NC_{—}000010.10).

Genomic DNA is typically used in the analysis of CYP2C19 alleles. Genomic DNA can be extracted from any biological sample containing nucleated cells, such as a peripheral blood sample or a tissue sample (e.g., mucosal scrapings of the lining of the mouth). Standard methods can be used to extract genomic DNA from a blood or tissue sample including, for example, phenol extraction. Alternatively, genomic DNA can be extracted with kits such as the QIAamp® Tissue Kit (Qiagen, Valencia, Calif.) and the Wizard® Genomic DNA purification kit (Promega, Madison, Wis.).

Any appropriate method can be used to identify a human as being heterozygous for CYP2C19*2 and heterozygous for CYP2C19*17. For example, standard genotyping and sequencing techniques can be used to determine that a human is heterozygous for CYP2C19*2 and heterozygous for CYP2C19*17.

Once identified as being heterozygous for CYP2C19*2 and heterozygous for CYP2C19*17, the human's genomic nucleic acid can be assessed to determine if CYP2C19*2 and CYP2C19*17 are in a cis or trans relationship. For example, samples that are heterozygous for CYP2C19*17 (c.-806C>T) and CYP2C19*2 (c.681G>A) in the CYP2C19 gene can be assessed using a cis/trans assay to determine if the CYP2C19*2 and CYP2C19*17 alleles occur on the same copy of the gene or on different copies. For each human to be assessed, the two copies of the CYP2C19 gene are amplified separately using either a wild type *17 PCR or a mutant *17 PCR with allele specific primers for the *17 allele. In some cases, the two copies of the CYP2C19 gene can be amplified separately using either a wild type c.-98 PCR or a mutant c.-98 PCR with allele specific primers for the c.-98 allele.

The created amplicons can be about 1000 base pairs in length. The two products can be purified using, for example, exonuclease and shrimp alkaline phosphatase to remove any unincorporated primers and dNTPs used in the PCR step. In some cases, the amplicons can be cleaned and used to perform a fluorescent DNA sequencing technique using big dye v3. Each PCR amplicon (two per a sample) can be set up with two or more (e.g., three) sequencing primers (for a total of six sequencing wells per a sample when three sequencing primers are used). One of the sequencing primers can be designed to sequence the *17 location and to verify that the allele specific primer amplified the proper allele. The other sequencing primer can be used to sequence the c.-98 position. In some cases, two sequencing primers can be used to bi-directionally sequence the c.-98 position.

For samples that are heterozygous for *17 and *2, the *2 position is located in exon 5 (about 19,000 bases away from the *17 position). Using the c.-98 variation (e.g., c.-98T>C), which is in linkage disequilibrium with the variation in the CYP2C19*2 position, the methods and materials provided herein can be used to determine if the mutant c.-98 (*2 associated SNP) is on the same copy as the *17 allele, based on which PCR product, wild type or mutant, the mutant c.-98 is found. If the c.-98 mutant is found on the mutant *17 PCR amplicon, then CYP2C19*2 and CYP2C19*17 are in cis. If the c.-98 mutant is found on the wild type *17 PCR amplicon, then CYP2C19*2 and CYP2C19*17 are in trans.

In some cases, the methods and materials provided herein can be used to assess a human heterozygous for CYP2C19*4 and heterozygous for CYP2C19*17 to determine if CYP2C19*4 and CYP2C19*17 are in cis or in trans.

The CYP2C19 polypeptide is an enzyme responsible for metabolizing a wide variety of drugs. Patients who have the CYP2C19*17 allele can make more of the CYP2C19 enzyme and can metabolize drugs faster than non-CYP2C19*17 humans. The CYP2C19*2 and CYP2C19*4 alleles are null alleles and can result in a normal amount of the CYP2C19 enzyme being made, but it is not able to metabolize drugs. If a human contains the mutant *17 and a null mutation (*2 or *4) on opposite alleles, then the human can make more of the functional CYP2C19 polypeptide. If the mutant *17 and the mutation of a null allele (*2 or *4) are on the same allele, then the human can make more of the non-functional CYP2C19 polypeptide and can metabolize less drug.

The invention will be further described in the following examples, which do not limit the scope of the invention described in the claims.

### EXAMPLES

### Example 1

### CYP2C19*2 and CYP2C19*17 Cis/Trans Assay by Dye Terminator Sequencing

Blood samples were obtained from 22 human patients who were confirmed to be heterozygous for CYP2C19*2 and heterozygous for CYP2C19*17. For each patient sample, the two copies of the CYP2C19 gene were amplified separately using either a wild type (WT)*17 polymerase chain reaction (PCR) procedure with WT allele specific primers for the *17 allele or a mutant (MT)*17 PCR procedure with MT allele specific primers for the *17 allele. In some cases, sequence specific PCR primers can be tagged (e.g., M13 tagged) to verify the specificity of the sequence specific primer when reviewing the sequence results. Briefly, genomic DNA was extracted from whole blood, and the CYP2C19 nucleic acid from each allele was amplified. The PCR master mix was prepared as described in Table 1.

TABLE 1
PCR master mix volumes per sample.

| | WT PCR | MT PCR |
|---|---|---|
| | Master | Master |
| | Mix | Mix |
| | Volume | Volume |
| | (μL/ | (μL/ |
| Reagent | sample) | sample) |
| DNAse/RNAse Free Water | 2.9 | 2.9 |
| FailSafe Buffer D (Epicentre) | 5 | 5 |
| 25 μM CYP2C19*17_SSPWT_F Primer | 0.5 | 0.0 |
| (5′-AATTTGTGTCTTCTGTTCTCAAAGC-3′; | | |
| SEQ ID NO: 1) | | |
| 25 μM CYP2C19*17_SSPMT_F Primer | 0.0 | 0.5 |
| (5′-AATTTGTGTCTTCTGTTCTCAAAGT-3′; | | |
| SEQ ID NO: 2) | | |
| 25 μM CYP2C19_EX1R_PCR Primer | 0.5 | 0.5 |
| (5′-ATACTTACATTGGTTAAGGATTTGCT- | | |
| GACA-3′; SEQ ID NO: 3) | | |
| FailSafe Enzyme (Epicentre) | 0.1 | 0.1 |

The master mix was vortexed and centrifuged. An aliquot of 9 μL of each master mix was added to 1 μL of the appropriate sample (patient sample or control). PCR was performed in an Applied Biosystems Thermal Cycler by holding at 95° C. for 2 minutes, then 30 cycles of denaturation at 95° C. for 30 seconds, annealing at 64° C. for 30 seconds, and extension at 72° C. for 1 minute, followed by a final extension at 72° C. for 10 minutes. A “No DNA” negative control was included with each multiplexed PCR reaction. This control was not sequenced. Absence of PCR amplification in the No DNA control was verified by Qiaxcel (QIAgen). Amplification of genetic material in the No DNA control identified contamination of reagent stocks and/or master mixes and served as a trigger for rejection of the batch.

A laboratory positive control (GM17109) was obtained by culture of a cell line from the Coriell cell repository. DNA was extracted from frozen cells at a concentration similar to blood (10,000 cells per mL). The positive control was PCR-amplified for each multiplex with every run.

The presence or absence of expected polymorphisms (star alleles) and variations was checked during review of each run (Table 2). Presence of expected polymorphisms and variations suggested a successful assay. Absence of expected variations suggested a problem with the assay.

TABLE 2
Expected Control Results.

| | Reported | Observed |
|---|---|---|
| Control | Genotype | Variations |
| GM17109 | *17, *1/*2 | *17 and *2 are in trans |
| | | (*17 MT PCR does not contain |
| | | the c.-98T>C variation) |

The PCR product was purified and sequenced in both directions using fluorescent dye-terminator chemistry. Briefly, the completed PCR reactions were vortexed and centrifuged. An ExoSAP enzyme mix (2.5 μL shrimp alkaline phosphatase (1 U/μL; USB) and 2.5 μL exonuclease I (10 U/μL; USB)) was added directly to each of the PCR products. These mixtures were vortexed, centrifuged, and incubated in a thermal cycler with heated lid as follows: 37° C. for 15 minutes, 80° C. for 15 minutes, and then held at 4° C.

Next, the samples were vortexed and centrifuged, and 45 μL of DNAse/RNAse free water was added to each ExoSap PCR product.

Each ExoSap PCR amplicon (2 per a sample) was set up with three sequencing primers (for a total of six sequencing wells per a sample). One primer sequenced the *17 location (verified the allele specific primer), and the other two primers were down stream and were used for bi-directional sequencing of a region upstream of exon 1 through the middle of exon 1. The sequencing reactions as outlined on Table 3 were prepared as follows: 6 μL of ExoSap PCR product, 1 μL of the 10 μM sequencing primer, 3 μL of Big Dye Mix (2.0 μL 5× buffer, 1.0 μL Big Dye (Big Dye Terminator V3.1 Cycle Sequencing kit, Applied Biosystems)).

TABLE 3
Sequencing reactions.

| Sample | Sample | | |
|---|---|---|---|
| 1 | 2 | Oligo | Sequencing Primer |
| WT-1A | WT-2A | 2C19_ | 5′-TTTAACCCCCTAAAAAAACACG-3′ |
| | | PRO*17R_ | (SEQ ID NO: 4) |
| | | PCR | |
| WT-1B | WT-2B | 2C19_-98F_ | 5′-GAGAACAAGACCAAAGGACATT-3′ |
| | | SEQ | (SEQ ID NO: 5) |
| WT-1C | WT-2C | 2C19_EX1R_ | 5′-GATATTTCCAATCACTGGGAGAG- |
| | | SEQ | GA-3′ (SEQ ID NO: 6) |
| MT-1A | MT-2A | 2C19_ | 5′-TTTAACCCCCTAAAAAAACACG-3′ |
| | | PRO*17R_ | (SEQ ID NO: 4) |
| | | PCR | |
| MT-1B | MT-2B | 2C19_-98F_ | 5′-GAGAACAAGACCAAAGGACATT-3′ |
| | | SEQ | (SEQ ID NO: 5) |
| MT-1C | MT-2C | 2C19_EX1R_ | 5′-GATATTTCCAATCACTGGGAG- |
| | | SEQ | AGGA-3′ (SEQ ID NO: 6) |

This sequencing reaction was performed using an Applied Biosystems Thermal Cycler with the following parameters: 95° C. for 2 minutes, followed by 23 cycles of 95° C. for 15 seconds, 50° C. for 15 seconds, 60° C. for 4 minutes, followed by a 4° C. hold. The sequencing product was purified using a 96-well Edge Biosystems filter plate with columns packed using rehydrated Sephadex G-50 fine powder. The 10 μL sequencing reaction was applied to the center of the packed column and centrifuged at 850×g for 5 minutes. For each well, 1 μL of the purified product was added to 9 μL of formamide and heat denatured at 95° C. for 2 minutes followed by an ice/water bath for 5 minutes. Finally, sequencing products were separated on an ABI 3130x1 (Applied Biosystems) automated sequencer, and trace files were analyzed for variations in the regions of interest using specialized mutation-detection software and visual inspection.

All 22 of the human patients heterozygous for CYP2C19*2 and heterozygous for CYP2C19*17 were found to have CYP2C19*2 and CYP2C19*17 in a trans relationship.

### Example 2

### CYP2C19*2 and CYP2C19*17 Cis/Trans Assay by Dye Terminator Sequencing

An assay similar to the assay described in Example 1 is performed to identify the cis or trans relationship of CYP2C19*2 and CYP2C19*17 for humans heterozygous for CYP2C19*2 and heterozygous for CYP2C19*17, with the exceptions that (a) the two copies of the CYP2C19 gene are amplified separately using either a wild type (WT) c.-98 polymerase chain reaction (PCR) procedure with WT allele specific primers for the c.-98 allele or a mutant (MT) c.-98 PCR procedure with MT allele specific primers for the c.-98 allele as opposed to using *17 specific primers, and (b) the sequencing reactions are designed to confirm the identity of the c.-98 sequence and identify the particular *17 sequences on the same allele as the MT and WT c.-98 sequences. The WT allele specific primers for the c.-98 allele is designed to have the following sequence: 5′-TGTGCCTCTTTGATGGATA-3′ (SEQ ID NO:7). The MT allele specific primers for the c.-98 allele is designed to have the following sequence: 5′-TGTGCCTCTTTGATGGATG-3′ (SEQ ID NO:8). Each of these c.-98 allele primers is used separately with the following forward primer: 5′-GGGCTGTTTTCCTTAGATAAA-3′ (SEQ ID NO:9).

The following sequencing primer is used to confirm the sequence present at the c.-98 location: 5′-GAGAACAAGACCAAAGGACATT-3′ (SEQ ID NO:5). The following sequencing primers are used to determine the sequence present at that *17 location: 5′-GTTGGTGCCACACAGCTCAT-3′ (SEQ ID NO:10) and 5′-TGGGAAAGGGAGACCCTGG-3′ (SEQ ID NO:11).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for assessing the cis or trans nature of a human heterozygous for CYP2C19*2 and heterozygous for CYP2C19*17, wherein said method comprises:
(a) determining if a CYP2C19 allele of said human comprises (i) a T, A, or G at the c.-806 position and a C, A, or G at the c.-98 position, (ii) a C at the c.-806 position and a T at the c.-98 position, (iii) a C at the c.-806 position and a C, A, or G at the c.-98 position, or (iv) a T, A, or G at the c.-806 position and a T at the c.-98 position,
(b) classifying said human as having CYP2C19*2 and CYP2C19*17 in cis if said CYP2C19 allele comprises (i) or (ii), and
(c) classifying said human as having CYP2C19*2 and CYP2C19*17 in trans if said CYP2C19 allele comprises (iii) or (iv).

2. The method of claim 1, wherein said human is a cardiovascular disease patient.

3. The method of claim 1, wherein said CYP2C19 allele comprises a T, A, or G at the c.-806 position and a C, A, or G at the c.-98 position, and said human is classified as having CYP2C19*2 and CYP2C19*17 in cis.

4. The method of claim 1, wherein said CYP2C19 allele comprises a T at the c.-806 position and a C at the c.-98 position, and said human is classified as having CYP2C19*2 and CYP2C19*17 in cis.

5. The method of claim 1, wherein said CYP2C19 allele comprises a C at the c.-806 position and a T at the c.-98 position, and said human is classified as having CYP2C19*2 and CYP2C19*17 in cis.

6. The method of claim 1, wherein said CYP2C19 allele comprises a C at the c.-806 position and a C, A, or G at the c.-98 position, and said human is classified as having CYP2C19*2 and CYP2C19*17 in trans.

7. The method of claim 1, wherein said CYP2C19 allele comprises a C at the c.-806 position and a C at the c.-98 position, and said human is classified as having CYP2C19*2 and CYP2C19*17 in trans.

8. The method of claim 1, wherein said CYP2C19 allele comprises a T, A, or G at the c.-806 position and a T at the c.-98 position, and said human is classified as having CYP2C19*2 and CYP2C19*17 in trans.

9. The method of claim 1, wherein said CYP2C19 allele comprises a T at the c.-806 position and a T at the c.-98 position, and said human is classified as having CYP2C19*2 and CYP2C19*17 in trans.

10. A method for identifying a human heterozygous for CYP2C19*2 and heterozygous for CYP2C19*17 having CYP2C19*2 and CYP2C19*17 in cis, wherein said method comprises:
(a) detecting the presence of an CYP2C19 allele of said human comprising (i) a T, A, or G at the c.-806 position and a C, A, or G at the c.-98 position or (ii) a C at the c.-806 position and a T at the c.-98 position, and
(b) classifying said human as having CYP2C19*2 and CYP2C19*17 in cis based at least in part on said presence of said CYP2C19 allele.

11. The method of claim 10, wherein said human is a cardiovascular disease patient.

12. The method of claim 10, wherein said CYP2C19 allele comprises a T, A, or G at the c.-806 position and a C, A, or G at the c.-98 position.

13. The method of claim 10, wherein said CYP2C19 allele comprises a T at the c.-806 position and a C at the c.-98 position.

14. The method of claim 10, wherein said CYP2C19 allele comprises a C at the c.-806 position and a T at the c.-98 position.

15. A method for identifying a human heterozygous for CYP2C19*2 and heterozygous for CYP2C19*17 having CYP2C19*2 and CYP2C19*17 in trans, wherein said method comprises:
(a) detecting the presence of an CYP2C19 allele of said human comprising (i) a C at the c.-806 position and a C, A, or G at the c.-98 position or (ii) a T, A, or G at the c.-806 position and a T at the c.-98 position, and
(b) classifying said human as having CYP2C19*2 and CYP2C19*17 in trans based at least in part on said presence of said CYP2C19 allele.

16. The method of claim 15, wherein said human is a cardiovascular disease patient.

17. The method of claim 15, wherein said CYP2C19 allele comprises a C at the c.-806 position and a C, A, or G at the c.-98 position.

18. The method of claim 15, wherein said CYP2C19 allele comprises a C at the c.-806 position and a C at the c.-98 position.

19. The method of claim 15, wherein said CYP2C19 allele comprises a T, A, or G at the c.-806 position and a T at the c.-98 position.

20. The method of claim 15, wherein said CYP2C19 allele comprises a T at the c.-806 position and a T at the c.-98 position.


SEQUENCE LISTING (11 sequences)

SEQ ID NO: 1 - DNA, 25 bp, Homo sapiens
aatttgtgtcttctgttctcaaagc

SEQ ID NO: 2 - DNA, 25 bp, Homo sapiens
aatttgtgtcttctgttctcaaagt

SEQ ID NO: 3 - DNA, 30 bp, Homo sapiens
atacttacattggttaaggatttgctgaca

SEQ ID NO: 4 - DNA, 22 bp, Homo sapiens
tttaaccccctaaaaaaacacg

SEQ ID NO: 5 - DNA, 22 bp, Homo sapiens
gagaacaagaccaaaggacatt

SEQ ID NO: 6 - DNA, 25 bp, Homo sapiens
gatatttccaatcactgggagagga

SEQ ID NO: 7 - DNA, 19 bp, Homo sapiens
tgtgcctctttgatggata

SEQ ID NO: 8 - DNA, 19 bp, Homo sapiens
tgtgcctctttgatggatg

SEQ ID NO: 9 - DNA, 21 bp, Homo sapiens
gggctgttttccttagataaa

SEQ ID NO: 10 - DNA, 20 bp, Homo sapiens
gttggtgccacacagctcat

SEQ ID NO: 11 - DNA, 18 bp, Homo sapiens
gggaaagggagaccctgg